(19) United States
(12) Patent Application Publication
(10) Pub. No.: US 2022/0160311 A1
(43) Pub. Date: May 26, 2022

(54) HEART RATE MEASUREMENT BASED ON REMOTE PHOTOPLETHYSMOGRAPHY

(71) Applicant: TENCENT AMERICA LLC, Palo Alto, CA (US)

(72) Inventors: Huang CHAO, Palo Alto, CA (US); Lianyi HAN, Palo Alto, CA (US); Hui TANG, Mountain View, CA (US); Shih-Yao LIN, Palo Alto, CA (US); Zhimin HUO, Pittsford, NY (US); Wei FAN, New York, NY (US)

(73) Assignee: TENCENT AMERICA LLC, Palo Alto, CA (US)

(21) Appl. No.: 17/103,128

(22) Filed: Nov. 24, 2020

(51) Int. Cl. A61B 5/00; A61B 5/024; G06K 9/00; G06K 9/32; G16H 30/20

(52) U.S. Cl. CPC A61B 5/7278; A61B 5/02427; A61B 5/7485; A61B 5/7203; A61B 2576/02; G06K 9/3233; G06K 9/00711; G16H 30/20; A61B 5/0077; G06K 9/00248

(57) ABSTRACT

An approach for determining a heart rate of a subject is performed by at least one processor and includes obtaining video data of the subject; detecting a face of the subject in the video data; selecting at least one region of interest (ROI) included in the face; acquiring photoplethysmography (PPG) signals based on the at least one ROI; and determining the heart rate of the subject based on the PPG signals

### BACKGROUND

Regular and noninvasive measurements of vital signs such as heart rate are important both in-hospital and at-home due to their fundamental role in the diagnosis of health conditions and monitoring of well-being. Currently, many techniques for vital sign measurement are based on contact sensors such as pulse oximeters and blood pressure cuffs. However, contact-based sensors are not convenient in some scenarios where physical contact is not preferred or even feasible.

### SUMMARY

According to some possible implementations, a method for determining a heart rate of a subject, is performed by at least one processor and includes obtaining video data of the subject; detecting a face of the subject in the video data; selecting at least one region of interest (ROI) included in the face; acquiring photoplethysmography (PPG) signals based on the at least one ROI; and determining the heart rate of the subject based on the PPG signals.

According to some possible implementations, a device for determining a heart rate of a subject includes a memory configured to store program code, and at least one processor configured to read the program code and operate as instructed by the program code, the program code including, including obtaining code configured to cause the at least one processor to obtain video data of the subject; detecting code configured to cause the at least one processor to detect a face of the subject in the video data; selecting code configured to cause the at least one processor to select at least one region of interest (ROI) included in the face; acquiring code configured to cause the at least one processor to acquire photoplethysmography (PPG) signals based on the at least one ROI; and determining code configured to cause the at least one processor to determine the heart rate of the subject based on the PPG signals.

According to some possible implementations, a non-transitory computer-readable medium stores instructions, the instructions including: one or more instructions that, when executed by one or more processors of a device, cause the one or more processors to: obtain video data of the subject; detect a face of the subject in the video data; select at least one region of interest (ROI) included in the face; acquire photoplethysmography (PPG) signals based on the at least one ROI; and determine the heart rate of the subject based on the PPG signals.

### BRIEF DESCRIPTION OF THE DRAWINGS

FIG. 1 is a diagram of an overview of an example implementation described herein;

FIG. 2 is a diagram of an example environment in which systems and/or methods, described herein, may be implemented;

FIG. 3 is a diagram of example components of one or more devices of FIG. 2; and

FIG. 4 is a flow chart of an example process for determining a heart rate of a subject.

### DETAILED DESCRIPTION

Embodiments relate to non-contact video-based physiological measurement which provides the potential for unobtrusive, concomitant measurement of important vital signs using ubiquitous and low-cost webcams or smartphone cameras.

Vital signs such as heart rate and oxygen saturation are currently measured using contact devices. However, non-contact methods for measuring vital signs are desirable both in hospital settings and for ubiquitous in-situ health tracking (e.g. on mobile phone and computers with webcams). This disclosure includes a method to measure heart rate based on the remote photoplethysmography (RPPG), which detects PPG signals from pixel changes in videos of human faces. This can be achieved with consumer-level cameras, such as webcams or mobile cameras, without any other external hardware. Under appropriate lighting conditions, the heart rate obtained from PRRG can be highly accurate compared to the contact measurements. This disclosure includes embodiments that pave the way to remotely and continuous monitor heart rate, and can be readily extended to measure and monitor other vital signs, such as breath rate, oxygen saturation etc.

For example, embodiments may provide non-contact measurement of heart rate. Currently, related art techniques to measure heart rate are based on contact sensors such as electrocardiogram (ECG) probes, chest straps, pulse oximeters and blood pressure cuffs. However, contact-based sensors are not convenient in all scenarios, e.g. contact sensors are known to cause skin damage in premature babies during their treatment in a neonatal intensive care unit (NICU). Embodiments may relate to non-contact methods for vital sign monitoring using a camera. Being non-contact, camera-based heart rate monitoring has many applications, from monitoring newborn babies in the NICU to in-situ continuous monitoring in everyday scenarios like working in front of a computer.

Further, embodiments may provide remote PPG. The two major challenges in estimating PPG using a camera are: (i) extremely low signal strength of the color-change signal, particularly for darker skin tones and/or under low lighting conditions, and (ii) motion artifacts due to an individual's movement in front of the camera. Therefore, related art camera-based heart rate monitoring may not perform well for subjects having darker skin tones and/or under low lighting conditions. Furthermore, related art algorithms may require a person to be nearly at rest facing a camera to ensure reliable measurements.

Embodiments may be used as a non-contact continuous monitoring heart changes over time that may reveal indications for health-related conditions, which otherwise may or may not be noticeable. Embodiments also may address the challenge of reliable heart rate estimation understand different environments, such as darker skin tones, low lighting conditions and different natural motions.

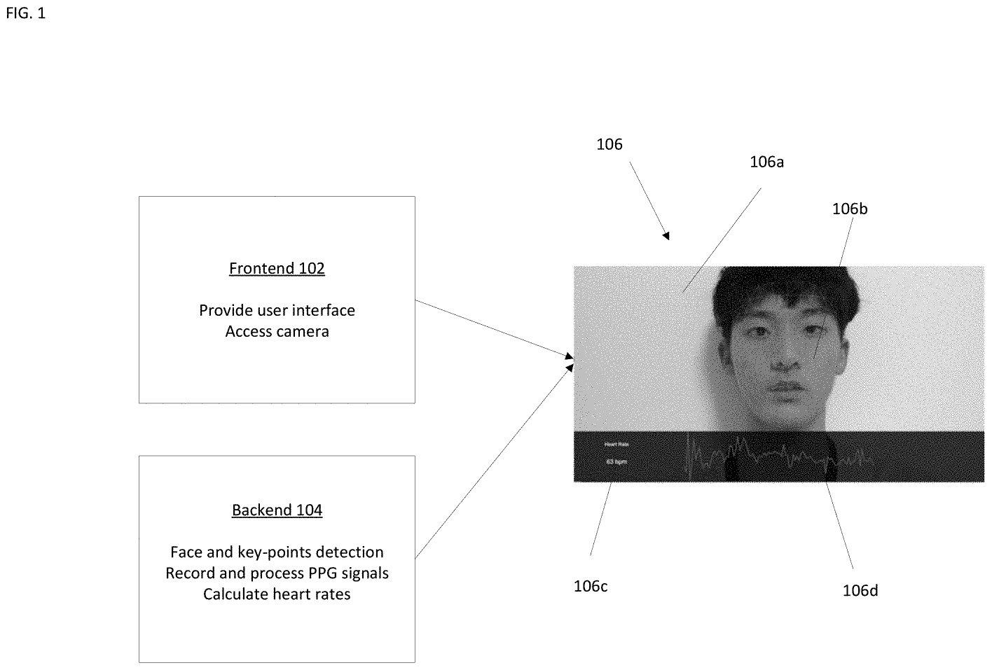

FIG. 1 is a diagram of an overview of an embodiment described herein. As shown in FIG. 1, a frontend 102 and a backend 104 may determine a heart rate of a subject and provide a determination result 106.

In embodiments, frontend 102 may access video data or image data from a camera. For example, frontend 102 may operate on a device which may include a camera, or which has access to image data or video data provided by a camera. In addition, frontend 102 may provide a user interface. In embodiments, the user interface of frontend 102 may provide or display determination result 106 to a user. In embodiments, the functions of frontend 102 described herein may be performed by one device or multiple devices.

In embodiments, backend 104 may analyze video data or image data provided to the backend 104, for example the video data or image data from the camera accessed by frontend 102, which may be provided by frontend 102 to backend 104. In embodiments, backend 104 may perform face detection on key-points detection. In embodiments, backend 104 may record and/or process PPG signals, and may calculate heart rates based on the PPG signals. In embodiments, the functions of backend 104 described herein may be performed by one device or multiple devices.

In embodiments, the functions of one or more of frontend 102 and backend 104 described herein may be performed by one device or multiple devices. In embodiments, one or more of frontend 102 and backend 104 may provide determination result 106.

In embodiments, determination result 106 may include, for example, one or more of an image 106a captured by the camera, a face detection result 106b, a textual indication 106c of the determined heart rate, and a graphical indication 106d of the heart rate. In embodiments, the graphical indication 106d may also indicate heart rate history, past heart rates, and/or a change in the heart rate over time. In embodiments, some or all of determination result 106 may be provided to frontend 102, backend 104, or any other device as desired, in the form of image data, video data, telemetry data, or any other type of data as desired.

### PPG Signal Acquisition

To extract PPG signals from videos of human faces, one or more faces may be detected in the videos first. To do that, face detection algorithms may be utilized, such as MediaPipe, which is an ultrafast face detection solution that comes with 6 landmarks and multi-face support. MediaPipe is based on BlazeFace, a lightweight and well-performing face detector tailored for mobile GPU inference. The detector's super-realtime performance may enable it to quickly locate an accurate facial region of interest (ROI) for PPG signal extraction. Because the regions of cheeks and forehead may have the strongest PPG signals, these regions may be chosen as ROI to acquire PPG signals, which can be done by averaging the RGB channel data over the pixels in each region.

### PPG Signal Processing and Heart Rate Calculation

To address the problems arising from weak or unstable PPG signals due to darker skin tones, low lighting conditions and different natural motions, the quality of the captured PPG signals may be assessed first, so that only properly sampled data may be selected for downstream processing. Because PPG signals may be periodic, the assessment of the data quality may be based on, for example, a number of peaks in a given period as well as the variance of the peaks/valleys. If the number of peaks is less than the expected number, then the signals in that period may be deemed to be low quality, and thus may be discarded. Likewise, if the variances of the peaks and valleys are larger than a certain threshold, those signals may also be rejected. The thresholds of the number of peaks and their variances may be empirically determined.

After assessing data quality, only stable data may be selected and subsequently processed, which may include for example denoising by using a band-pass (0.5-5 Hz) Butterworth filter. The processed data may then be used to calculate heart rate values using the peak-find algorithm, and the heart rate values calculated from different color channels and regions may be averaged to estimate the final heart rate.

For example, multiple heart rate values may be determined based on, for example, any combination of a red color channel of a cheek ROI, a green color channel of a cheek ROT, a blue color channel of a cheek ROI, a red color channel of a forehead ROT, a green color channel of a forehead ROI, and a blue color channel of a forehead ROI, and these multiple heart rate values may be averaged to determine a final heart rate value.

An advantage of remote PPG may be to provide a method for non-invasive and passive monitoring of heart rate. This could be useful for medical circumstances where physical contact with the patient is not preferred. Moreover, as a video camera could capture multiple persons during a single video, heart rate monitoring of multiple persons could be possible with the same configuration. Moreover, the method could be applied to detect psychological disorders/anomalies using the long term changes in other vital signs detected by the same methodology.

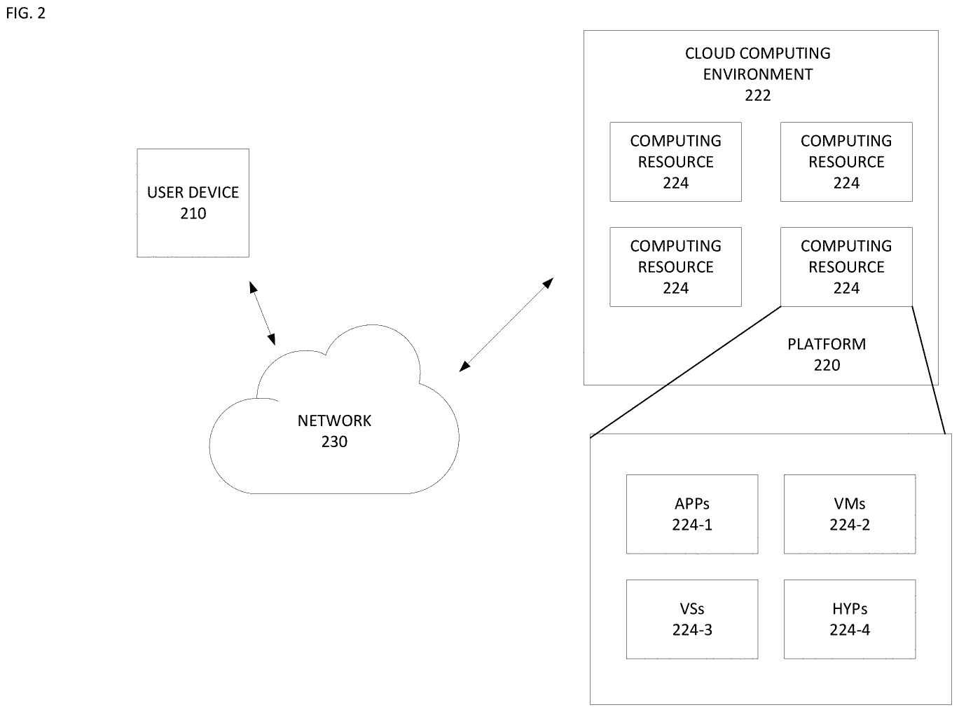

FIG. 2 is a diagram of an example environment 200 in which systems and/or methods, described herein, may be implemented. As shown in FIG. 2, environment 200 may include a user device 210, a platform 220, and a network 230. Devices of environment 200 may interconnect via wired connections, wireless connections, or a combination of wired and wireless connections. In embodiments, any of the functions of frontend 102 and backend 104 may be performed by any combination of elements illustrated in FIG. 2. For example, in embodiments, user device 210 may perform one or more functions associated with frontend 102, and platform 220 may perform one or more functions associated with backend 104.

User device 210 includes one or more devices capable of receiving, generating, storing, processing, and/or providing information associated with platform 220. For example, user device 210 may include a computing device (e.g., a desktop computer, a laptop computer, a tablet computer, a handheld computer, a smart speaker, a server, etc.), a mobile phone (e.g., a smart phone, a radiotelephone, etc.), a wearable device (e.g., a pair of smart glasses or a smart watch), or a similar device. In some implementations, user device 210 may receive information from and/or transmit information to platform 220.

Platform 220 includes one or more devices capable of determining a heart rate of a subject using RPPG, as described elsewhere herein. In some implementations, platform 220 may include a cloud server or a group of cloud servers. In some implementations, platform 220 may be designed to be modular such that certain software components may be swapped in or out depending on a particular need. As such, platform 220 may be easily and/or quickly reconfigured for different uses.

In some implementations, as shown, platform 220 may be hosted in cloud computing environment 222. Notably, while implementations described herein describe platform 220 as being hosted in cloud computing environment 222, in some implementations, platform 220 is not be cloud-based (i.e., may be implemented outside of a cloud computing environment) or may be partially cloud-based.

Cloud computing environment 222 includes an environment that hosts platform 220. Cloud computing environment 222 may provide computation, software, data access, storage, etc. services that do not require end-user (e.g., user device 210) knowledge of a physical location and configuration of system(s) and/or device(s) that hosts platform 220. As shown, cloud computing environment 222 may include a group of computing resources 224 (referred to collectively as “computing resources 224” and individually as “computing resource 224”).

Computing resource 224 includes one or more personal computers, workstation computers, server devices, or other types of computation and/or communication devices. In some implementations, computing resource 224 may host platform 220. The cloud resources may include compute instances executing in computing resource 224, storage devices provided in computing resource 224, data transfer devices provided by computing resource 224, etc. In some implementations, computing resource 224 may communicate with other computing resources 224 via wired connections, wireless connections, or a combination of wired and wireless connections.

As further shown in FIG. 2, computing resource 224 includes a group of cloud resources, such as one or more applications (“APPs”) 224-1, one or more virtual machines (“VMs”) 224-2, virtualized storage (“VSs”) 224-3, one or more hypervisors (“HYPs”) 224-4, or the like.

Application 224-1 includes one or more software applications that may be provided to or accessed by user device 210. Application 224-1 may eliminate a need to install and execute the software applications on user device 210. For example, application 224-1 may include software associated with platform 220 and/or any other software capable of being provided via cloud computing environment 222. In some implementations, one application 224-1 may send/receive information to/from one or more other applications 224-1, via virtual machine 224-2.

Virtual machine 224-2 includes a software implementation of a machine (e.g., a computer) that executes programs like a physical machine. Virtual machine 224-2 may be either a system virtual machine or a process virtual machine, depending upon use and degree of correspondence to any real machine by virtual machine 224-2. A system virtual machine may provide a complete system platform that supports execution of a complete operating system (“OS”). A process virtual machine may execute a single program, and may support a single process. In some implementations, virtual machine 224-2 may execute on behalf of a user (e.g., user device 210), and may manage infrastructure of cloud computing environment 222, such as data management, synchronization, or long-duration data transfers.

Virtualized storage 224-3 includes one or more storage systems and/or one or more devices that use virtualization techniques within the storage systems or devices of computing resource 224. In some implementations, within the context of a storage system, types of virtualizations may include block virtualization and file virtualization. Block virtualization may refer to abstraction (or separation) of logical storage from physical storage so that the storage system may be accessed without regard to physical storage or heterogeneous structure. The separation may permit administrators of the storage system flexibility in how the administrators manage storage for end users. File virtualization may eliminate dependencies between data accessed at a file level and a location where files are physically stored. This may enable optimization of storage use, server consolidation, and/or performance of non-disruptive file migrations.

Hypervisor 224-4 may provide hardware virtualization techniques that allow multiple operating systems (e.g., “guest operating systems”) to execute concurrently on a host computer, such as computing resource 224. Hypervisor 224-4 may present a virtual operating platform to the guest operating systems, and may manage the execution of the guest operating systems. Multiple instances of a variety of operating systems may share virtualized hardware resources.

Network 230 includes one or more wired and/or wireless networks. For example, network 230 may include a cellular network (e.g., a fifth generation (5G) network, a long-term evolution (LTE) network, a third generation (3G) network, a code division multiple access (CDMA) network, etc.), a public land mobile network (PLMN), a local area network (LAN), a wide area network (WAN), a metropolitan area network (MAN), a telephone network (e.g., the Public Switched Telephone Network (PSTN)), a private network, an ad hoc network, an intranet, the Internet, a fiber optic-based network, or the like, and/or a combination of these or other types of networks.

The number and arrangement of devices and networks shown in FIG. 2 are provided as an example. In practice, there may be additional devices and/or networks, fewer devices and/or networks, different devices and/or networks, or differently arranged devices and/or networks than those shown in FIG. 2. Furthermore, two or more devices shown in FIG. 2 may be implemented within a single device, or a single device shown in FIG. 2 may be implemented as multiple, distributed devices. Additionally, or alternatively, a set of devices (e.g., one or more devices) of environment 200 may perform one or more functions described as being performed by another set of devices of environment 200.

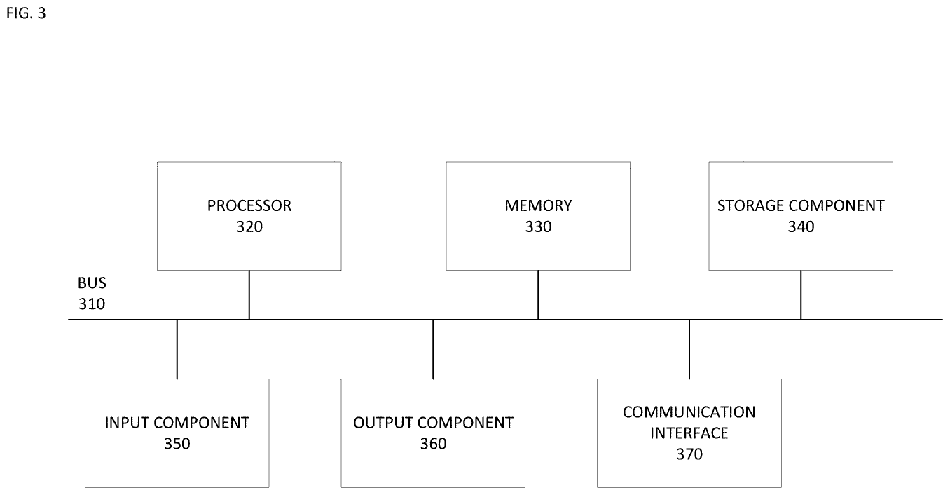

FIG. 3 is a diagram of example components of a device 300. Device 300 may correspond to user device 210 and/or platform 220. As shown in FIG. 3, device 300 may include a bus 310, a processor 320, a memory 330, a storage component 340, an input component 350, an output component 360, and a communication interface 370.

Bus 310 includes a component that permits communication among the components of device 300. Processor 320 is implemented in hardware, firmware, or a combination of hardware and software. Processor 320 is a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component. In some implementations, processor 320 includes one or more processors capable of being programmed to perform a function. Memory 330 includes a random access memory (RAM), a read only memory (ROM), and/or another type of dynamic or static storage device (e.g., a flash memory, a magnetic memory, and/or an optical memory) that stores information and/or instructions for use by processor 320.

Storage component 340 stores information and/or software related to the operation and use of device 300. For example, storage component 340 may include a hard disk (e.g., a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive.

Input component 350 includes a component that permits device 300 to receive information, such as via user input (e.g., a touch screen display, a keyboard, a keypad, a mouse, a button, a switch, and/or a microphone). Additionally, or alternatively, input component 350 may include a sensor for sensing information (e.g., a global positioning system (GPS) component, an accelerometer, a gyroscope, and/or an actuator). Output component 360 includes a component that provides output information from device 300 (e.g., a display, a speaker, and/or one or more light-emitting diodes (LEDs)).

Communication interface 370 includes a transceiver-like component (e.g., a transceiver and/or a separate receiver and transmitter) that enables device 300 to communicate with other devices, such as via a wired connection, a wireless connection, or a combination of wired and wireless connections. Communication interface 370 may permit device 300 to receive information from another device and/or provide information to another device. For example, communication interface 370 may include an Ethernet interface, an optical interface, a coaxial interface, an infrared interface, a radio frequency (RF) interface, a universal serial bus (USB) interface, a Wi-Fi interface, a cellular network interface, or the like.

Device 300 may perform one or more processes described herein. Device 300 may perform these processes in response to processor 320 executing software instructions stored by a non-transitory computer-readable medium, such as memory 330 and/or storage component 340. A computer-readable medium is defined herein as a non-transitory memory device. A memory device includes memory space within a single physical storage device or memory space spread across multiple physical storage devices.

Software instructions may be read into memory 330 and/or storage component 340 from another computer-readable medium or from another device via communication interface 370. When executed, software instructions stored in memory 330 and/or storage component 340 may cause processor 320 to perform one or more processes described herein. Additionally, or alternatively, hardwired circuitry may be used in place of or in combination with software instructions to perform one or more processes described herein. Thus, implementations described herein are not limited to any specific combination of hardware circuitry and software.

The number and arrangement of components shown in FIG. 3 are provided as an example. In practice, device 300 may include additional components, fewer components, different components, or differently arranged components than those shown in FIG. 3. Additionally, or alternatively, a set of components (e.g., one or more components) of device 300 may perform one or more functions described as being performed by another set of components of device 300.

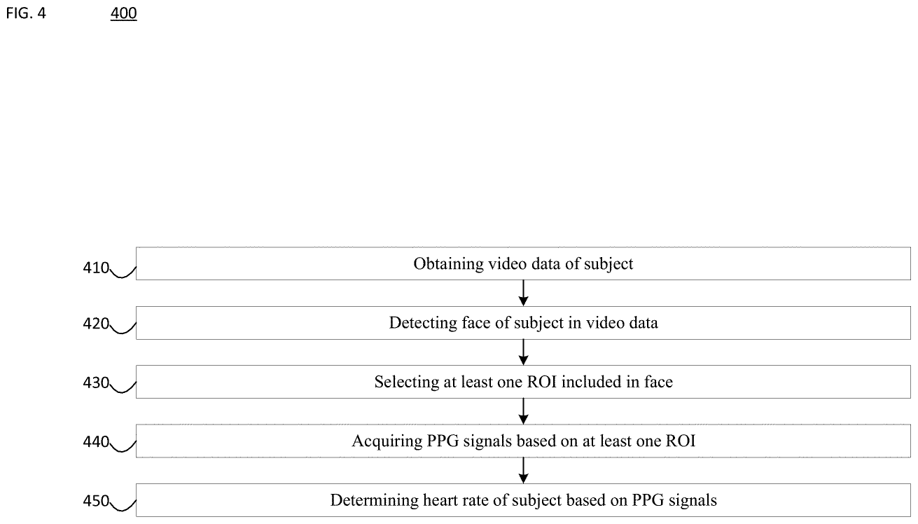

FIG. 4 is a flow chart of an example process 400 for determining a heart rate of a subject. In some implementations, one or more process blocks of FIG. 4 may be performed by platform 220. In some implementations, one or more process blocks of FIG. 4 may be performed by another device or a group of devices separate from or including platform 220, such as user device 210.

As shown in FIG. 4, process 400 may include obtaining video data of the subject (block 410).

As further shown in FIG. 4, process 400 may include detecting a face of the subject in the video data (block 420).

As further shown in FIG. 4, process 400 may include selecting at least one region of interest (ROI) included in the face (block 430).

As further shown in FIG. 4, process 400 may include acquiring photoplethysmography (PPG) signals based on the at least one ROI (block 440).

As further shown in FIG. 4, process 400 may include determining the heart rate of the subject based on the PPG signals (block 450).

In embodiments, the face may be detected using at least one of a MediaPipe face detection algorithm or a BlazeFace face detection algorithm.

In embodiments, the at least one ROI may include at least one of a forehead included in the face or a cheek included in the face.

In embodiments, the PPG signals may be obtained from among detected signals based on at least one threshold value corresponding to number of peaks in the PPG signals.

In embodiments, the at least one threshold value may include an upper threshold value and a lower threshold value.

In embodiments, the PPG signals may be obtained from among the detected signals by discarding first signals having a first number peaks higher than the upper threshold value, and by discarding second signals having a second number of peaks lower than the lower threshold value.

In embodiments, the heart rate may be determined by applying a peak-find algorithm to the PPG signals.

In embodiments, the PPG signals may include a plurality of PPG signals acquired based on a plurality of color channels and a plurality of ROIs.

In embodiments, the heart rate may be determined by averaging the plurality of PPG signals.

Although FIG. 4 shows example blocks of process 400, in some implementations, process 400 may include additional blocks, fewer blocks, different blocks, or differently arranged blocks than those depicted in FIG. 4. Additionally, or alternatively, two or more of the blocks of process 400 may be performed in parallel.

The foregoing disclosure provides illustration and description, but is not intended to be exhaustive or to limit the implementations to the precise form disclosed. Modifications and variations are possible in light of the above disclosure or may be acquired from practice of the implementations.

As used herein, the term component is intended to be broadly construed as hardware, firmware, or a combination of hardware and software.

It will be apparent that systems and/or methods, described herein, may be implemented in different forms of hardware, firmware, or a combination of hardware and software. The actual specialized control hardware or software code used to implement these systems and/or methods is not limiting of the implementations. Thus, the operation and behavior of the systems and/or methods were described herein without reference to specific software code—it being understood that software and hardware may be designed to implement the systems and/or methods based on the description herein.

Even though particular combinations of features are recited in the claims and/or disclosed in the specification, these combinations are not intended to limit the disclosure of possible implementations. In fact, many of these features may be combined in ways not specifically recited in the claims and/or disclosed in the specification. Although each dependent claim listed below may directly depend on only one claim, the disclosure of possible implementations includes each dependent claim in combination with every other claim in the claim set.

No element, act, or instruction used herein should be construed as critical or essential unless explicitly described as such. Also, as used herein, the articles “a” and “an” are intended to include one or more items, and may be used interchangeably with “one or more.” Furthermore, as used herein, the term “set” is intended to include one or more items (e.g., related items, unrelated items, a combination of related and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

What is claimed is:

1. A method for determining a heart rate of a subject, the method performed by at least one processor and comprising:
obtaining video data of the subject;
detecting a face of the subject in the video data;
selecting at least one region of interest (ROI) included in the face;
acquiring photoplethysmography (PPG) signals based on the at least one ROI; and
determining the heart rate of the subject based on the PPG signals.

2. The method of claim 1, wherein the face is detected using at least one of a MediaPipe face detection algorithm or a BlazeFace face detection algorithm.

3. The method of claim 1, wherein the at least one ROI includes at least one of a forehead included in the face or a cheek included in the face.

4. The method of claim 1, wherein the PPG signals are obtained from among detected signals based on at least one threshold value corresponding to number of peaks in the PPG signals.

5. The method of claim 4, wherein the at least one threshold value comprises an upper threshold value and a lower threshold value, and
wherein the PPG signals are obtained from among the detected signals by discarding first signals having a first number peaks higher than the upper threshold value, and by discarding second signals having a second number of peaks lower than the lower threshold value.

6. The method of claim 1, wherein the heart rate is determined by applying a peak-find algorithm to the PPG signals.

7. The method of claim 1, wherein the PPG signals comprise a plurality of PPG signals acquired based on a plurality of color channels and a plurality of ROIs, and wherein the heart rate is determined by averaging the plurality of PPG signals.

8. A device for determining a heart rate of a subject, comprising:
at least one memory configured to store program code;
at least one processor configured to read the program code and operate as instructed by the program code, the program code including:
obtaining code configured to cause the at least one processor to obtain video data of the subject;
detecting code configured to cause the at least one processor to detect a face of the subject in the video data;
selecting code configured to cause the at least one processor to select at least one region of interest (ROI) included in the face;
acquiring code configured to cause the at least one processor to acquire photoplethysmography (PPG) signals based on the at least one ROI; and
determining code configured to cause the at least one processor to determine the heart rate of the subject based on the PPG signals.

9. The device of claim 8, wherein the face is detected using at least one of a MediaPipe face detection algorithm or a BlazeFace face detection algorithm.

10. The device of claim 8, wherein the at least one ROI includes at least one of a forehead included in the face or a cheek included in the face.

11. The device of claim 8, wherein the PPG signals are obtained from among detected signals based on at least one threshold value corresponding to number of peaks in the PPG signals.

12. The device of claim 11, wherein the at least one threshold value comprises an upper threshold value and a lower threshold value, and
wherein the PPG signals are obtained from among the detected signals by discarding first signals having a first number peaks higher than the upper threshold value, and by discarding second signals having a second number of peaks lower than the lower threshold value.

13. The device of claim 8, wherein the heart rate is determined by applying a peak-find algorithm to the PPG signals.

14. The device of claim 8, wherein the PPG signals comprise a plurality of PPG signals acquired based on a plurality of color channels and a plurality of ROIs, and
wherein the heart rate is determined by averaging the plurality of PPG signals.

15. A non-transitory computer-readable medium storing instructions, the instructions comprising: one or more instructions that, when executed by one or more processors of a device for determining a heart rate of a subject, cause the one or more processors to:
obtain video data of the subject;
detect a face of the subject in the video data;
select at least one region of interest (ROI) included in the face;
acquire photoplethysmography (PPG) signals based on the at least one ROI; and
determine the heart rate of the subject based on the PPG signals.

16. The non-transitory computer-readable medium of claim 15, wherein the at least one ROI includes at least one of a forehead included in the face or a cheek included in the face.

17. The non-transitory computer-readable medium of claim 15, wherein the PPG signals are obtained from among detected signals based on at least one threshold value corresponding to number of peaks in the PPG signals.

18. The non-transitory computer-readable medium of claim 17, wherein the at least one threshold value comprises an upper threshold value and a lower threshold value, and
wherein the PPG signals are obtained from among the detected signals by discarding first signals having a first number peaks higher than the upper threshold value, and by discarding second signals having a second number of peaks lower than the lower threshold value.

19. The non-transitory computer-readable medium of claim 15, wherein the heart rate is determined by applying a peak-find algorithm to the PPG signals.

20. The non-transitory computer-readable medium of claim 15, wherein the PPG signals comprise a plurality of PPG signals acquired based on a plurality of color channels and a plurality of ROIs, and
wherein the heart rate is determined by averaging the plurality of PPG signals.
